(19) United States
(12) Patent Application Publication
(10) Pub. No.: US 2024/0138803 A1
(43) Pub. Date: May 2, 2024

(54) ATRIAL FIBRILLATION DETECTION USING HEART SOUND MORPHOLOGY

(71) Applicant: Cardiac Pacemakers, Inc., St. Paul, MN (US)

(72) Inventors: Kaylen Yeri Kang, Annandale, VA (US); Jonathan Bennett Shute, Eagan, MN (US); Mojgan Goftari, Shoreview, MN (US); Abhijit Rajan, Shoreview, MN (US)

(21) Appl. No.: 18/500,637

(22) Filed: Nov. 2, 2023

Related U.S. Application Data
(60) Provisional application No. 63/421,834, filed on Nov. 2, 2022.

(51) Int. Cl. A61B 7/00; A61B 5/352; A61B 5/361

(52) U.S. Cl. CPC A61B 7/00; A61B 5/352; A61B 5/361; A61B 2562/0204

(57) ABSTRACT

Systems and methods are disclosed to determine an indication of one of an atrial fibrillation S4 heart sound or a non-atrial fibrillation S4 heart sound for an S4 signal portion of cardiac acceleration information of a patient based on determined first and second correlations of a morphology of the S4 signal portion to atrial-fibrillation and non-atrial fibrillation S4 templates, respectively, and to determine an atrial fibrillation event of the patient using cardiac electrical information of the patient and the determined indication for the S4 signal portion.

### CLAIM OF PRIORITY

This application claims the benefit of U.S. Provisional Application No. 63/421,834, filed on Nov. 2, 2022, which is hereby incorporated by reference in its entirety.

### TECHNICAL FIELD

This document relates generally to medical devices and more particularly to atrial fibrillation detection using heart sound morphology.

### BACKGROUND

Heart failure (HF) is a reduction in the ability of the heart to deliver enough blood to meet bodily needs. Heart failure patients commonly have enlarged hearts with weakened cardiac muscles, resulting in reduced contractility and poor cardiac output. Signs of heart failure include pulmonary congestion, edema, difficulty breathing, etc. Heart failure is often a chronic condition, but can also occur suddenly, affecting the left, right, or both sides of the heart. Causes of heart failure include coronary artery disease, myocardial infarction, high blood pressure, atrial fibrillation, valvular heart disease, alcoholism, infection, cardiomyopathy, or one or more other conditions leading to a decreased pumping efficiency of the heart.

An arrhythmia is an abnormal heart rhythm (e.g., fast, slow, irregular, etc.). Arrhythmias include, among others, bradycardia, tachycardia, premature, extra, or skipped heart beats, and atrial or ventricular fibrillation affecting one or more chambers of the heart. Atrial fibrillation (AF) is as an abnormal heart rhythm characterized by rapid and irregular activity in the left or right atria of the heart. Atrial fibrillation is commonly associated with a reduction in cardiac output, an increased risk of heart failure, dementia, and stroke. Risk factors for atrial fibrillation include, among others, high blood pressure, heart failure, valvular heart disease, chronic obstructive pulmonary disorder (COPD), obesity, and sleep apnea.

Ambulatory medical devices (AMDs), including implantable, subcutaneous, wearable, or one or more other medical devices, etc., can monitor, detect, or treat various conditions, including heart failure, atrial fibrillation, etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation. Accurate identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of ambulatory medical devices, and reduce healthcare costs.

### SUMMARY

Systems and methods are disclosed to determine an indication of one of an atrial fibrillation S4 heart sound or a non-atrial fibrillation S4 heart sound for an S4 signal portion of cardiac acceleration information of a patient based on determined first and second correlations of a morphology of the S4 signal portion to atrial-fibrillation and non-atrial fibrillation S4 templates, respectively, and to determine an atrial fibrillation event of the patient using cardiac electrical information of the patient and the determined indication for the S4 signal portion.

An example (e.g., “Example 1”) of subject matter (e.g., a medical device system) may comprise a signal receiver circuit configured to receive (1) cardiac electrical information of a patient, including a timing metric between first and second cardiac features of the patient; and (2) cardiac acceleration information of the patient, including a fourth heart sound (S4) signal portion occurring between the first and second cardiac features of the patient; an assessment circuit configured to determine a first correlation of a morphology of the S4 signal portion to a non-atrial fibrillation S4 template and a second correlation of a morphology of the S4 signal portion to an atrial fibrillation S4 template, and to determine an indication of one of an atrial fibrillation S4 heart sound or a non-atrial fibrillation S4 heart sound for the S4 signal portion based on the determined first and second correlations; and an atrial fibrillation detection circuit configured to detect an atrial fibrillation event of the patient using the received timing metric and the determined indication for the S4 heart sound portion.

In Example 2, the subject matter of Example 1 may optionally be configured to include a cardiac sensor, coupled to the signal receiver circuit, configured to sense the cardiac electrical information of the patient and a heart sound sensor, coupled to the signal receiver circuit, configured to sense the cardiac acceleration information of the patient.

In Example 3, the subject matter of any one or more of Examples 1-2 may optionally be configured to include an implantable medical device comprising the electrical sensor, the heart sound sensor, the signal receiver circuit, the assessment circuit, and the atrial fibrillation detection circuit.

In Example 4, the subject matter of any one or more of Examples 1-3 may optionally be configured such that the cardiac sensor comprises a processing circuit configured to determine the timing metric between the first and second cardiac features of a first cardiac interval of the patient or between first and second cardiac features of respective successive first and second cardiac intervals of the patient and the heart sound sensor comprises a processing circuit configured to determine the S4 signal portion in an S4 window of the cardiac acceleration information of the corresponding first or second cardiac interval of the patient.

In Example 5, the subject matter of any one or more of Examples 1˜4 may optionally be configured such that the first and second cardiac features include equivalent detected features in successive first and second cardiac intervals.

In Example 6, the subject matter of any one or more of Examples 1-5 may optionally be configured such that the first cardiac feature is an R wave of a first cardiac interval and the second cardiac feature is an R wave of an ensuing second cardiac interval.

In Example 7, the subject matter of any one or more of Examples 1-6 may optionally be configured such that the timing metric is a timing metric of a cardiac interval, and wherein the S4 signal portion is an S4 signal portion of the cardiac interval.

In Example 8, the subject matter of any one or more of Examples 1-7 may optionally be configured such the atrial fibrillation event comprises multiple cardiac intervals and the assessment circuit is configured to determine the indication of one of the atrial fibrillation S4 heart sound or the non-atrial fibrillation S4 heart sound for the S4 signal portion based on the determined first and second correlations for each of the multiple cardiac intervals.

In Example 9, the subject matter of any one or more of Examples 1-8 may optionally be configured such that the atrial fibrillation event comprises multiple cardiac intervals, the assessment circuit is configured to determine a composite S4 signal portion using S4 signal portions occurring over the multiple cardiac intervals, the assessment circuit is configured to determine the first correlation between the composite S4 signal portion and the non-atrial fibrillation S4 template and the second correlation between the composite S4 signal portion and the atrial fibrillation S4 heart sound, and the assessment circuit is configured to determine the indication of one of the atrial fibrillation S4 heart sound or the non-atrial fibrillation S4 heart sound for the composite S4 signal portion based on the determined first and second correlations.

In Example 10, the subject matter of any one or more of Examples 1-9 may optionally be configured such that the assessment circuit is configured to determine the indication of one of the atrial fibrillation S4 heart sound or the non-atrial fibrillation S4 heart sound for the S4 heart sound in the S4 signal portion based on a difference between a correlation of the S4 signal portion to the non-atrial fibrillation S4 template and a correlation of the S4 signal portion to the atrial fibrillation S4 template.

In Example 11, the subject matter of any one or more of Examples 1-10 may optionally be configured such that the assessment circuit is configured to determine the indication of one of the atrial fibrillation S4 heart sound or the non-atrial fibrillation S4 heart sound for the S4 heart sound in the S4 signal portion based on a difference of the correlation of the S4 signal portion the atrial fibrillation S4 template from the correlation of the S4 signal portion to the non-atrial fibrillation S4 template.

An example (e.g., “Example 12”) of subject matter (e.g., a method) may comprise: receiving, at a signal receiver circuit, cardiac electrical information of a patient, including a timing metric between first and second cardiac features of the patient; receiving, at the signal receiver circuit, cardiac acceleration information of the patient, including a fourth heart sound (S4) signal portion occurring between the first and second cardiac features of the patient; determining, using an assessment circuit, a first correlation of a morphology of the S4 signal portion to a non-atrial fibrillation S4 template and a second correlation of the morphology of the S4 signal portion to an atrial fibrillation S4 template; determining, using the assessment circuit, an indication of one of an atrial fibrillation S4 heart sound or a non-atrial fibrillation S4 heart sound for the S4 signal portion based on the determined first and second correlations; and detecting, using an atrial fibrillation detection circuit, an atrial fibrillation event of the patient using the received timing metric and the determined indication for the S4 signal portion.

In Example 13, the subject matter of Example 12 may optionally be configured to include sensing the cardiac electrical information of the patient using a cardiac sensor coupled to the signal receiver circuit and sensing the cardiac acceleration information of the patient using a heart sound sensor coupled to the signal receiver circuit.

In Example 14, the subject matter of any one or more of Examples 12-13 may optionally be configured such that the electrical sensor, the heart sound sensor, the signal receiver circuit, the assessment circuit, and the atrial fibrillation detection circuit are components of an implantable medical device.

In Example 15, the subject matter of any one or more of Examples 12-14 may optionally be configured to include determining, using a processing circuit of the cardiac sensor, the timing metric between the first and second cardiac features of a first cardiac interval of the patient or between first and second cardiac features of respective successive first and second cardiac intervals of the patient and determining, using a processing circuit of the heart sound sensor, the S4 signal portion in an S4 window of the cardiac acceleration information of the corresponding first or second cardiac interval of the patient.

In Example 16, the subject matter of any one or more of Examples 12-15 may optionally be configured such that the first and second cardiac features include equivalent detected features in successive first and second cardiac intervals.

In Example 17, the subject matter of any one or more of Examples 12-16 may optionally be configured such that the timing metric is a timing metric of a first cardiac interval, and wherein the S4 signal portion is an S4 signal portion of the first cardiac interval.

In Example 18, the subject matter of any one or more of Examples 12-17 may optionally be configured such that the atrial fibrillation event comprises multiple cardiac intervals and determining the indication of one of the atrial fibrillation S4 heart sound or the non-atrial fibrillation S4 heart sound for the S4 signal portion includes based on the determined first and second correlations for each of the multiple cardiac intervals.

In Example 19, the subject matter of any one or more of Examples 12-18 may optionally be configured such that the atrial fibrillation event comprises multiple cardiac intervals, wherein the method includes determining a composite S4 signal portion using S4 signal portions occurring over the multiple cardiac intervals, wherein determining the first correlation comprises between the composite S4 signal portion and the non-atrial fibrillation S4 template, determining the second correlation comprises between the composite S4 signal portion and the atrial fibrillation S4 heart sound, and determining the indication of one of the atrial fibrillation S4 heart sound or the non-atrial fibrillation S4 heart sound comprises for the composite S4 signal portion based on the determined first and second correlations.

In Example 20, the subject matter of any one or more of Examples 12-19 may optionally be configured such that determining the indication of one of the atrial fibrillation S4 heart sound or the non-atrial fibrillation S4 heart sound for the S4 heart sound in the S4 signal portion comprises based on a difference between a correlation of the S4 signal portion to the non-atrial fibrillation S4 template and a correlation of the S4 signal portion to the atrial fibrillation S4 template.

In Example 21, subject matter (e.g., a system or apparatus) may optionally combine any portion or combination of any portion of any one or more of Examples 1-20 to comprise “means for” performing any portion of any one or more of the functions or methods of Examples 1-20, or at least one “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a relationship between measured patient physiologic information including patient heart sound information and cardiac electrical information over a cardiac cycle.

FIG. 2 illustrates generally example performance differences for evaluation of a fourth heart sound (S4) signal using different features.

FIG. 3 illustrates an example atrial fibrillation and non-atrial fibrillation S4 signal templates.

FIGS. 4-6 illustrate example false P wave determinations made using cardiac electrical information.

FIG. 7 illustrates example aggregate cardiac electrical information composite signals.

FIG. 8 illustrates example aggregate fourth heart sound (S4) signal portion composite signals.

FIG. 9 illustrates an example system to determine an atrial fibrillation event using heart sound morphology.

FIG. 10 illustrates an example patient management system and portions of an environment in which the patient management system may operate.

FIG. 11 illustrates an example method of determining an atrial fibrillation event of a patient using heart sound morphology.

FIG. 12 illustrates a block diagram of an example machine upon which any one or more of the techniques discussed herein may perform.

### DETAILED DESCRIPTION

Implantable and ambulatory medical devices can include, or be configured to receive cardiac electrical information from, one or more electrodes located within, on, or proximate to the heart, such as coupled to a lead and located in one or more chambers of the heart or within the vasculature of the heart near one or more chambers. Ambulatory medical devices can additionally include or be configured to receive mechanical acceleration information from one or more accelerometer sensors to determine and monitor patient acceleration information, such as cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiration rate, phase, breathing sounds, etc.), etc.

Arrhythmia events, including potential arrhythmia events, such as atrial fibrillation events or potential events, can be detected using sensed or received cardiac electrical information, including, for example, detected atrial or ventricular events (e.g., beats, r-waves, p-waves, etc.) or intervals therebetween occurring within a detection window, often between 30 seconds and 2 minutes, though in certain examples longer or shorter. Ambulatory medical devices can determine, for example, using timing information between events, in certain examples, in combination with one or more other detected events, whether atrial fibrillation is present or not in each detection window, and can additionally determine to store or transmit sensed or detected information, such as for transmission to a remote device, based on the determination. In certain examples, the ambulatory medical device can aggregate information from multiple sensors, detect various events using information from each sensor separately or in combination, update a detection status based on the information, and transmit a message or an alert to one or more remote devices that a detection has been made, that information has been stored or transmitted, such that one or more additional processes or systems can use the stored or transmitted detection or information for one or more other review or processes.

Atrial fibrillation detection algorithms commonly rely on cardiac electrical information features, such as cardiac intervals between successive R waves, individual beat-to-beat rate variance, etc. Examples of atrial fibrillation detection algorithms, including various atrial fibrillation detection parameters and criteria, can be found, for example, in the commonly assigned Krueger et al. U.S. patent application Ser. No. 14/825,669, titled “Atrial Fibrillation Detection Using Ventricular Rate Variability” (herein, “the '669 application”); Perschbacher et al. U.S. patent application Ser. No. 15/082,440, titled “Atrial Fibrillation Detection” (herein, “the '440 application”); Krueger et al. U.S. patent application Ser. No. 15/341,565, titled “Method and Apparatus for Enhancing Ventricular Based Atrial Fibrillation Detection Using Atrial Activity” (herein, “the '565 application”); and Perschbacher et al. U.S. patent application Ser. No. 15/864,953, titled “Atrial Fibrillation Discrimination Using Heart Rate Clustering” (herein, “the '953 application”), each of which are hereby incorporated by reference in their entireties, including their disclosure of atrial fibrillation detection and atrial fibrillation detection algorithms including, for example: atrial fibrillation detection using pairs of ventricular information detected from a ventricle, including rate changes and rate change characteristics, and determination of valid heart beats or intervals using various characteristics, including threshold rates, intervals, morphology criterion, etc., such as disclosed in the '669 application; atrial fibrillation detection using a distribution of ventricular depolarization intervals, such as disclosed in the '440 application; atrial fibrillation detection using atrial activity scores from an atrial detection window prior to a detected ventricular polarization, such as disclosed in the '565 application; atrial fibrillation discrimination using clustered depolarization information, such as disclosed in the '953 application, etc.

Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

The S4 heart sound, which reflects atrial contraction during sinus rhythm, is frequently not present during atrial fibrillation. Accordingly, in certain examples, a determination of whether the S4 heart sound is present or absent can be used to improve determinations of atrial fibrillation. For example, S4 morphology can be been used to determine if the S4 heart sound is present in an S4 window within a specific cardiac cycle, such as disclosed in the commonly assigned Thakur et al. U.S. patent application Ser. No. 16/215,230, titled “Systems And Methods For Detecting Atrial Tachyarrhythmia Using Heart Sounds” (herein, “the '230 application”), hereby incorporated by reference in its entirety, including its disclosure of comparing a fourth heart sound (S4) signal portion to an S4 template and determining if a matching score exceeds a threshold value, and using the determined score to improve determinations of atrial fibrillation.

The present inventors have recognized, among other things, additional improvements to determinations of atrial fibrillation using S4 morphology. For example, in certain patients, at least some heart sound signal is present in an S4 window, including in certain examples at least some S4 signal portion, during atrial fibrillation, such that presence or absence alone may not provide the most accurate determination of atrial fibrillation. Accordingly, to improve sensitivity and specificity of atrial fibrillation detection, separate specific atrial fibrillation and non-atrial fibrillation S4 signal templates can be determined, and the S4 signal portion of a heart sound signal can be compared to both specific and separate atrial fibrillation and non-atrial fibrillation S4 signal templates and a resulting atrial fibrillation determination can be made based on both comparisons, such as, for example:

score=corr(data(t),nonAF_model)−corr(data(t),AF_model (1)

The score can include a determination of whether the S4 signal portion is more accurately representative of an S4 signal portion during non-atrial fibrillation (e.g., normal sinus rhythm) or an S4 signal portion during atrial fibrillation, in contrast to a gross determination of absence or presence alone. For example, a positive score can be indicative of a non-atrial fibrillation S4 heart sound and a negative score can be indicative of an atrial fibrillation S4 heart sound. The term “corr” can be a correlation function configured to determine a similarity between two signals, “data(t)” can be the S4 signal portion of a heart sound signal, “nonAF_model” can be the non-atrial fibrillation S4 signal template, and “AF_model” can be the atrial fibrillation S4 signal template.

In other examples, the determination can additionally include other fiducials or patterns or spectral content of the S4 signal portion, such as described herein. Improved sensitivity and specificity of atrial fibrillation determination can improve detection of false positive atrial fibrillation episodes, more accurately controlling an active sensing or data storage mode (e.g., sampling time, sampling frequency, length of stored episodes, etc.) of ambulatory medical devices or sensors associated with such determinations, reducing storage of false positive atrial fibrillation episodes, reducing data transmission of stored episodes to one or more remote devices, reducing human review of such transmitted and determined events or episodes, or providing or changing one or more therapy parameters to the patient based on the detected events or determinations. In certain examples, initial detections of atrial fibrillation events can be rejected or confirmed based on the positive and negative scores, respectively. In other examples, data can be relabeled, triggered storage can be reversed, mode transitions can be reversed, etc., based on the determined positive or negative scores, etc.

In cardiac electrical information (e.g., ECG) based atrial fibrillation detection alone (e.g., without heart sounds or P wave confirmation), P waves are only detected in 30-35% of false atrial fibrillation detections. While P wave confirmation can improve false positive atrial fibrillation detections in contrast to cardiac electrical information based detection alone, and the presence of the S4 heart sound can provide a separate indication of the presence of a P wave in light of misdetection, such as from artifacts, noise, vector or lead placement, or specific patient anatomy, the present inventors have recognized that determination of separate correlations to separate specific atrial fibrillation and non-atrial fibrillation S4 signal templates, such as described herein, can further improve performance of atrial fibrillation detection. In particular, in one example, 140 of 163 clinically determined ECG-based false positives were corrected by the techniques described herein.

A technological problem exists in medical devices that in low-power monitoring modes, ambulatory medical devices powered by one or more rechargeable or non-rechargeable batteries have to make certain tradeoffs between battery life, or in the instance of implantable medical devices with non-rechargeable batteries, between device replacement periods often including surgical procedures, and sampling resolution, sampling periods, and processing, storage, and transmission of sensed physiologic information. Medical devices can include higher-power monitoring modes. Physiologic information, such as indicative of a potential adverse physiologic event, can be used to transition from a low-power monitoring mode (e.g., a low-power mode) to a higher-power or higher-resolution monitoring mode (e.g., a high-power mode). In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiological information detected in the low-power mode indicates a possible event, valuable information has been lost, unable to be recorded in the high-power mode.

The inverse is also true, in that false or inaccurate determinations that trigger a high-power mode unnecessarily unduly limit the usable life of certain ambulatory medical devices. The change in modes can enable higher resolution sampling or an increase in the sampling frequency or number or types of sensors used to sense physiologic information leading up to and including the potential event. For example, heart sounds and patient activity are often detected using non-overlapping time periods of the same, single- or multi-axis accelerometer, at different sampling frequencies and power costs. In one example, the transition to a high-power mode can include using the accelerometer to detect heart sounds throughout the high-power mode, or at a larger percentage of the high-power mode than during a corresponding low-power mode, etc. Additionally, waveforms for medical events are often recorded, stored in long-term memory, and frequently transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device. Accordingly, for numerous reasons, it is advantageous to accurately detect and determine physiologic events, including reducing false positive device detections, to properly manage and utilize medical device resources.

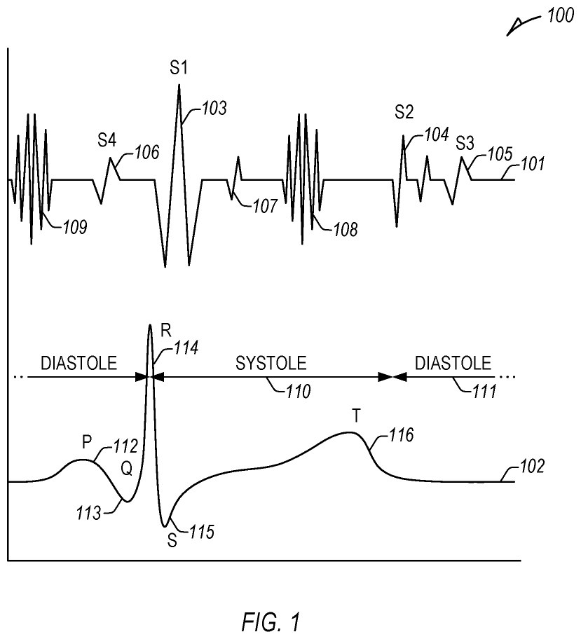

FIG. 1 illustrates a relationship 100 between measured patient physiologic information including patient heart sound information 101 (including a first heart sound (S1) 103, a second heart sound (S2) 104, a third heart sound (S3) 105, a fourth heart sound (S4) 106, ejection sounds 107, systolic murmurs 108, opening sounds between S2 and S3, and diastolic murmurs 109) and cardiac electrical information 102 (including P, Q, R, S, and T waves 112-116 of an electrocardiogram signal) over a cardiac cycle, including periods of systole 110 and diastole 111.

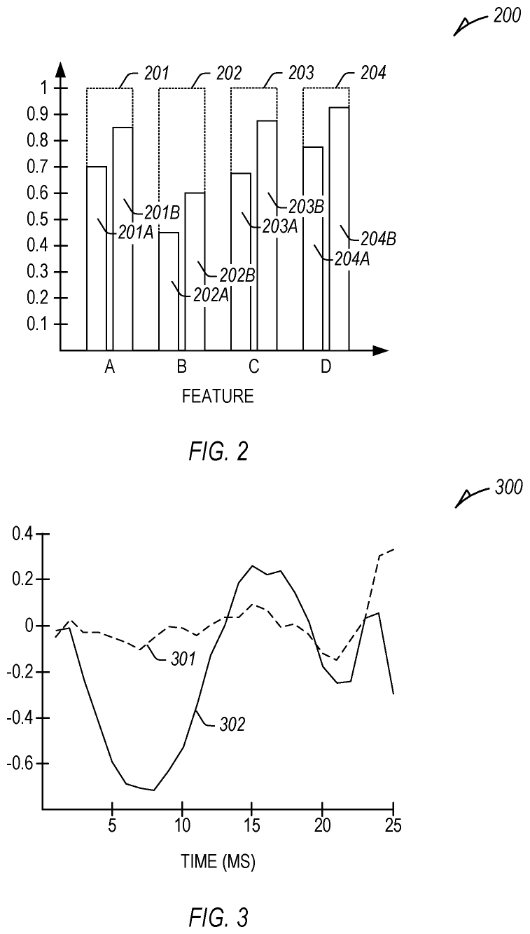

FIG. 2 illustrates generally example performance differences 200 for evaluation of an S4 signal using different features, including: (A) ECG-based detection 201 and respective sensitivity 201A and specificity 201B; (B) root-means squared (RMS) determination of heart sound energy in an S4 detection window 202 and respective sensitivity 202A and specificity 202B; (C) raw heart sound signal correlation for a single cardiac interval 203 and respective sensitivity 203A and specificity 203B; and (D) filtered and de-noised correlation for an S4 signal portion 204 and respective sensitivity 204A and specificity 204B.

FIG. 3 illustrates an example heart sound templates 300 in an S4 heart sound window, the templates 300 including an atrial fibrillation S4 signal template 301 and a non-atrial fibrillation S4 signal template 302 (e.g., an S4 signal template in normal sinus rhythm). In certain examples, the atrial and non-atrial fibrillation S4 signal templates 301, 302 can be population templates determined based on clinical determinations of S4 signal portions of heart sound waveforms of patients displaying atrial fibrillation and non-atrial fibrillation (e.g., normal sinus rhythm) respectively. The atrial and non-atrial fibrillation signal templates 301, 302 can be generated as a mean or median of multiple pre-labeled (e.g., clinically labeled or adjudicated as either atrial fibrillation signal portions or non-atrial fibrillation signal portions, such as by a clinician, separate detection algorithm, etc.). In certain examples, the labels can include cardiac electrical information based atrial fibrillation detection algorithms collected and reviewed over time, such as from a patient or a population of patients, etc. In certain examples, the respective signal templates can be updated as more determinations are collected and made available.

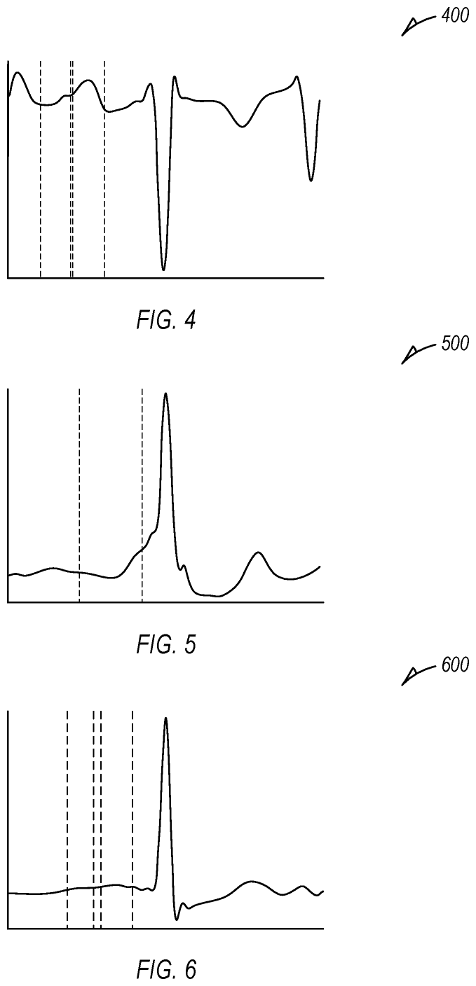

FIGS. 4-6 illustrate example false P wave determinations, demonstrating challenges in accurately detecting P waves across various patients using cardiac electrical information. FIG. 4 illustrates an example atrial flutter rhythm 400 detected as a false P wave detection. FIG. 5 illustrates an example rhythm 500 including an artifact (e.g., noise) masking a P wave detection. FIG. 6 illustrates an example intrinsically lower P wave 600 due to a patient detection vector or anatomy masking a P wave detection.

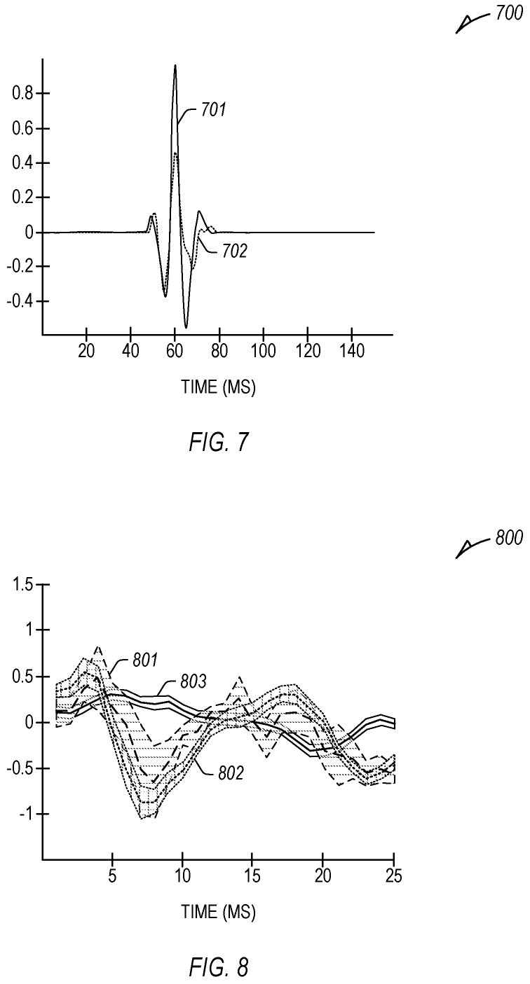

FIG. 7 illustrates example aggregate cardiac electrical information composite signals 700 including true positive P wave detections 701 and false positive P wave detections 702. Composite signals generally include combinations (e.g., a mean or median representation, in certain examples filtered, etc.) of multiple signals. The similarities between the true positive and false positive composite signals illustrates the difficulties in distinguishing between the different events across patient populations.

FIG. 8 illustrates example aggregate S4 signal portion composite signals 800 including true negative atrial fibrillation detections 801, false positive atrial fibrillation detections 802 (e.g., from cardiac electrical information based atrial fibrillation detection), and positive atrial fibrillation detections 803. The positive atrial fibrillation detections 803 show a substantial difference from the true negative atrial fibrillation detections 801 and the false positive atrial fibrillation detections 802. However, true negative atrial fibrillation detections 801 and false positive atrial fibrillation detections 802 are more similar, illustrating more subtle variation in the cardiac mechanical signal.

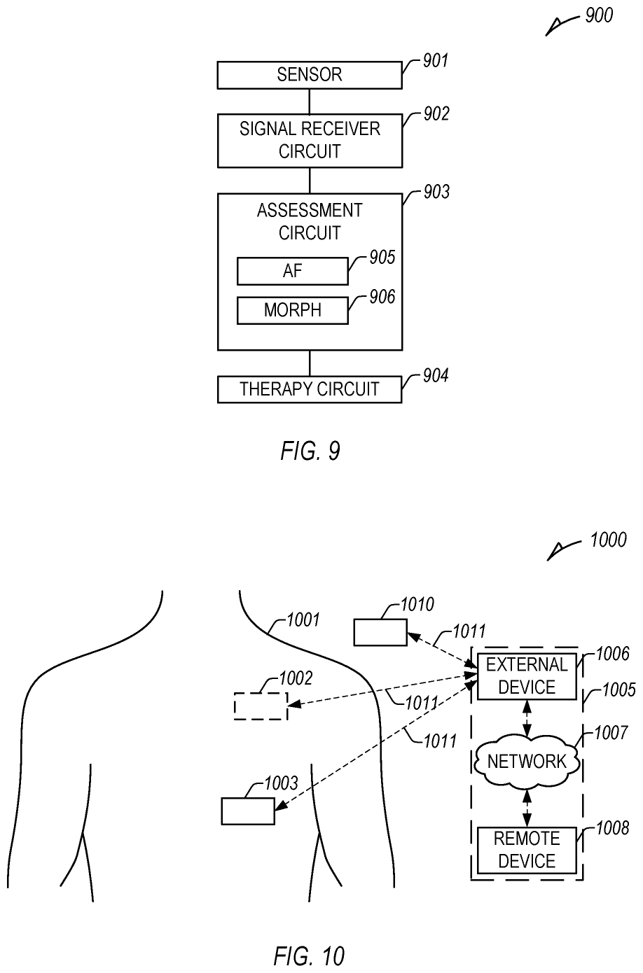

FIG. 9 illustrates an example system 900 to detect an atrial fibrillation event using heart sound morphology, such as by determining an indication of one of an atrial fibrillation S4 heart sound or a non-atrial fibrillation S4 heart sound for an S4 signal portion of cardiac acceleration information of a patient based on determined first and second correlations of a morphology of the S4 signal portion to atrial-fibrillation and non-atrial fibrillation S4 templates, respectively, and determining an atrial fibrillation event of the patient using cardiac electrical information of the patient and the determined indication for the S4 signal portion.

The example system 900 can include a medical-device system, a cardiac rhythm management (CRM) device, etc. In an example, one or more aspects of the example system 900 can be a component of, or communicatively coupled to, an ambulatory medical device (AMD), an insertable cardiac monitor, etc. The system 900 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 900 can include a single medical device or a plurality of medical devices implanted in a patient's body or otherwise positioned on or about the patient to monitor patient physiologic information of the patient using one or more sensors, such as a sensor 901. In an example, the sensor 901 can include one or more of: a respiration sensor configured to receive respiration information (e.g., a respiration rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., intrathoracic impedance sensor, transthoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 900 can include a signal receiver circuit 902 and an assessment circuit 903. The signal receiver circuit 902 can be configured to receive physiologic information of a patient (or group of patients) from the sensor 901. The assessment circuit 903 can be configured to receive information from the signal receiver circuit 902, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 903 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

The assessment circuit 903 can be configured to provide an output to a user, such as to a display or one or more other user interface, the output including a score, a trend, an alert, or other indication. In other examples, the assessment circuit 903 can be configured to provide an output to another circuit, machine, or process, such as a therapy circuit 904 (e.g., a cardiac resynchronization therapy (CRT) circuit, a chemical therapy circuit, etc.), etc., to control, adjust, or cease a therapy of a medical device, a drug delivery system, etc., or otherwise alter one or more processes or functions of one or more other aspects of a medical-device system, such as one or more cardiac resynchronization therapy parameters, drug delivery, dosage determinations or recommendations, etc. In an example, the therapy circuit 904 can include one or more of a stimulation control circuit, a cardiac stimulation circuit, a neural stimulation circuit, a dosage determination or control circuit, etc. In other examples, the therapy circuit 904 can be controlled by the assessment circuit 903, or one or more other circuits, etc.

In certain examples, the assessment circuit 903 can include, among other circuits, an atrial fibrillation detection circuit 905 and a morphology circuit 906. The atrial fibrillation detection circuit 905 can be configured to perform one or more atrial fibrillation detection algorithms or otherwise determine one or more indications of atrial fibrillation in one or more cardiac cycles or a time window comprising a plurality of cardiac cycles, such as to determine one or more measures and to compare the one or more measures to a patient-specific or population threshold. The morphology circuit 906 can be configured to determine a correlation (e.g., a cross-correlation, a correlation coefficient, correlation waveform analysis, etc.) between a shape of one or more signal features, such as an S4 signal portion of a heart sound signal of one or more cardiac cycles to one or more templates, such as described herein, such as to determine one or more measures and to compare the one or more measures to a patient-specific or population threshold. In certain examples, the one or more templates can be patient-specific or population templates. An indication of one of an atrial fibrillation S4 heart sound or a non-atrial fibrillation S4 heart sound in the S4 signal portion can be determined based on a difference between determined correlations between the S4 signal portion of the heart sound signal and both of the non-atrial fibrillation S4 template and the atrial fibrillation S4 template.

In other examples, the determination can additionally include other fiducials or patterns or spectral content of the S4 signal portion. For example, the magnitude of the frequency content in S4 window can be used to determine the indication of one of the atrial fibrillation S4 heart sound or the non-atrial fibrillation S4 heart sound. Generally, an atrial fibrillation S4 heart sound will have less frequency content in an S4 window than a non-atrial fibrillation heart sound. Accordingly, template frequency content for each of the atrial fibrillation S4 heart sound and non-atrial fibrillation S4 heart sound can be determined, either population or patient-specific templates, and such templates can be used as an additional feature in the determinations described herein.

Changes in the S4 signal portion over time can be indicative of changes in ventricular stiffening. In an example, an increase in the S4 signal portion over time can be used to detect worsening heart condition. Relative changes beyond a threshold can trigger an alert, notification, mode switch, or one or more other medical device changes or actions, such as described herein.

FIG. 10 illustrates an example patient management system 1000 and portions of an environment in which the patient management system 1000 may operate. The patient management system 1000 can perform a range of activities, including remote patient monitoring and diagnosis of a disease condition. Such activities can be performed proximal to a patient 1001, such as in a patient home or office, through a centralized server, such as in a hospital, clinic, or physician office, or through a remote workstation, such as a secure wireless mobile computing device.

The patient management system 1000 can include one or more ambulatory medical devices, an external system 1005, and a communication link 1011 providing for communication between the one or more ambulatory medical devices and the external system 1005. The one or more ambulatory medical devices can include an implantable medical device (AVID) 1002, a wearable medical device 1003, or one or more other implantable, leadless, subcutaneous, external, wearable, or ambulatory medical devices configured to monitor, sense, or detect information from, determine physiologic information about, or provide one or more therapies to treat various conditions of the patient 1001, such as one or more cardiac or non-cardiac conditions (e.g., dehydration, sleep disordered breathing, etc.).

In an example, the implantable medical device 1002 can include one or more traditional cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 1001. In another example, the implantable medical device 1002 can include a monitor implanted, for example, subcutaneously in the chest of patient 1001, the implantable medical device 1002 including a housing containing circuitry and, in certain examples, one or more sensors, such as a temperature sensor, etc.

Traditional cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 1002 can include an assessment circuit configured to detect or determine specific physiologic information of the patient 1001, or to determine one or more conditions or provide information or an alert to a user, such as the patient 1001 (e.g., a patient), a clinician, or one or more other caregivers or processes, such as described herein. The implantable medical device 1002 can alternatively or additionally be configured as a therapeutic device configured to treat one or more medical conditions of the patient 1001. The therapy can be delivered to the patient 1001 via the lead system and associated electrodes or using one or more other delivery mechanisms. The therapy can include delivery of one or more drugs to the patient 1001, such as using the implantable medical device 1002 or one or more of the other ambulatory medical devices, etc. In some examples, therapy can include cardiac resynchronization therapy for rectifying dyssynchrony and improving cardiac function in heart failure patients. In other examples, the implantable medical device 1002 can include a drug delivery system, such as a drug infusion pump to deliver drugs to the patient for managing arrhythmias or complications from arrhythmias, hypertension, hypotension, or one or more other physiologic conditions. In other examples, the implantable medical device 1002 can include one or more electrodes configured to stimulate the nervous system of the patient or to provide stimulation to the muscles of the patient airway, etc.

The wearable medical device 1003 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 1005 can include a dedicated hardware/software system, such as a programmer, a remote server-based patient management system, or alternatively a system defined predominantly by software running on a standard personal computer. The external system 1005 can manage the patient 1001 through the implantable medical device 1002 or one or more other ambulatory medical devices connected to the external system 1005 via a communication link 1011. In other examples, the implantable medical device 1002 can be connected to the wearable medical device 1003, or the wearable medical device 1003 can be connected to the external system 1005, via the communication link 1011. This can include, for example, programming the implantable medical device 1002 to perform one or more of acquiring physiologic data, performing at least one self-diagnostic test (such as for a device operational status), analyzing the physiologic data, or optionally delivering or adjusting a therapy for the patient 1001. Additionally, the external system 1005 can send information to, or receive information from, the implantable medical device 1002 or the wearable medical device 1003 via the communication link 1011. Examples of the information can include real-time or stored physiologic data from the patient 1001, diagnostic data, such as detection of patient hydration status, hospitalizations, responses to therapies delivered to the patient 1001, or device operational status of the implantable medical device 1002 or the wearable medical device 1003 (e.g., battery status, lead impedance, etc.). The communication link 1011 can be an inductive telemetry link, a capacitive telemetry link, or a radio-frequency (RF) telemetry link, or wireless telemetry based on, for example, “strong” Bluetooth or IEEE 602.11 wireless fidelity “Wi-Fi” interfacing standards. Other configurations and combinations of patient data source interfacing are possible.

The external system 1005 can include an external device 1006 in proximity of the one or more ambulatory medical devices, and a remote device 1008 in a location relatively distant from the one or more ambulatory medical devices, in communication with the external device 1006 via a communication network 1007. Examples of the external device 1006 can include a medical device programmer. The remote device 1008 can be configured to evaluate collected patient or patient information and provide alert notifications, among other possible functions. In an example, the remote device 1008 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 1008 can receive data from multiple patients. The data can be collected by the one or more ambulatory medical devices, among other data acquisition sensors or devices associated with the patient 1001. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 1008 may additionally include one or more locally configured clients or remote clients securely connected over the communication network 1007 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 1008, including the server and the interconnected clients, may also execute a follow-up scheme by sending follow-up requests to the one or more ambulatory medical devices, or by sending a message or other communication to the patient 1001 (e.g., the patient), clinician or authorized third party as a compliance notification.

The communication network 1007 can provide wired or wireless interconnectivity. In an example, the communication network 1007 can be based on the Transmission Control Protocol/Internet Protocol (TCP/IP) network communication specification, although other types or combinations of networking implementations are possible. Similarly, other network topologies and arrangements are possible.

One or more of the external device 1006 or the remote device 1008 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 1006 or the remote device 1008 can include a respective display unit for displaying the physiologic or functional signals, or alerts, alarms, emergency calls, or other forms of warnings to signal the detection of arrhythmias. In some examples, the external system 1005 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 1005 can be implemented using hardware, software, firmware, or combinations thereof. Portions of the one or more ambulatory medical devices or the external system 1005 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit a network interface and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 1010 can be configured to send information to or receive information from one or more of the ambulatory medical devices or the external system 1005 using the communication link 1011. In an example, the one or more ambulatory medical devices, the external device 1006, or the remote device 1008 can be configured to control one or more parameters of the therapy device 1010. The external system 1005 can allow for programming the one or more ambulatory medical devices and can receives information about one or more signals acquired by the one or more ambulatory medical devices, such as can be received via a communication link 1011. The external system 1005 can include a local external implantable medical device programmer. The external system 1005 can include a remote patient management system that can monitor patient status or adjust one or more therapies such as from a remote location.

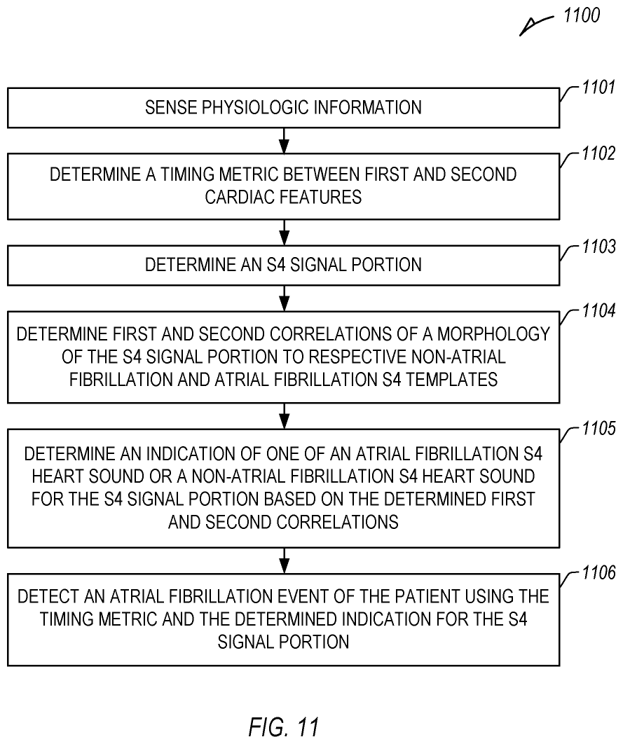

FIG. 11 illustrates an example method 1100 of determining an atrial fibrillation event of a patient using heart sound morphology, such as by determining an indication of one of an atrial fibrillation S4 heart sound or a non-atrial fibrillation S4 heart sound for an S4 signal portion of cardiac acceleration information of a patient based on determined first and second correlations of a morphology of the S4 signal portion to atrial-fibrillation and non-atrial fibrillation S4 templates, respectively, and determining an atrial fibrillation event of the patient using cardiac electrical information of the patient and the determined indication for the S4 signal portion.

At 1101, physiologic information of a patient can be sensed, such as by one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.).

At 1102, a timing metric between first and second cardiac features can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

At 1103, an S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

At 1104, first and second correlations of a morphology or shape of the S4 signal portion to respective non-atrial fibrillation and atrial fibrillation S4 templates can be determined, such as using an assessment circuit to determine measures of similarity between the different signals. At 1105, an indication of one of an atrial fibrillation S4 heart sound or a non-atrial fibrillation S4 heart sound for the S4 signal portion can be determined based on the determined first and second correlations, such as using the assessment circuit or one or more other processing circuits to determine a difference between the determined first and second correlations.

At 1106, an atrial fibrillation event of the patient can be determined using the received timing metric and the determined indication for the S4 signal portion, such as using an atrial fibrillation detection circuit. In an example, an initial determination of atrial fibrillation can be determined using cardiac electrical information alone, such as based on heart rate or cardiac interval timing between successive cardiac cycles or intervals or groups of cardiac cycles. An initial determination of atrial fibrillation based on the cardiac electrical information can trigger determining the S4 signal portion of the cardiac acceleration information, sensing the cardiac acceleration information (e.g., over the S4 window of one or more cardiac intervals), or determination of the first or second correlations or determination of the indication of one of an atrial fibrillation S4 heart sound or non-atrial fibrillation S4 heart sound for the S4 signal portion based on the determined first and second correlations.

In certain examples, the determined atrial fibrillation event can comprise multiple cardiac intervals, in certain example, occurring over a specific time interval (e.g., a threshold beat-to-beat timing or rate variation or pattern occurring over a 2-minute time window, etc.). In an example, the indication of one of the atrial fibrillation S4 heart sound or the non-atrial fibrillation S4 heart sound for the S4 signal portion can be determined based on the determined first and second correlations for each of the multiple cardiac intervals.

In an example, a composite S4 signal portion can be determined using S4 signal portions occurring over multiple cardiac intervals (e.g., combining multiple signal portions into a representative composite signal), and the first and second correlations can be determined between the composite S4 signal portion and the non-atrial fibrillation S4 template and the atrial fibrillation S4 heart sound, respectively. The indication of one of the atrial fibrillation S4 heart sound or the non-atrial fibrillation S4 heart sound can be determined for the composite S4 signal portion based on the determined first and second correlations.

In an example, the indication of one of the atrial fibrillation S4 heart sound or the non-atrial fibrillation S4 heart sound for the S4 heart sound in the S4 signal portion can be determined based on a difference between a correlation of the S4 signal portion to the non-atrial fibrillation S4 template and a correlation of the S4 signal portion to the atrial fibrillation S4 template. In other examples, the indication of one of the atrial fibrillation S4 heart sound or the non-atrial fibrillation S4 heart sound for the S4 heart sound in the S4 signal portion can be determined based on a difference of the correlation of the S4 signal portion the atrial fibrillation S4 template from the correlation of the S4 signal portion to the non-atrial fibrillation S4 template.

In an example, the indication of one of the atrial fibrillation S4 heart sound or the non-atrial fibrillation S4 heart sound for the S4 heart sound in the S4 signal portion can additionally be determined based on one or more heart sound parameters. In an example, a heart sound parameter can include one or more of an S1, S2, S3, or S4 value, such as an amplitude or energy value (e.g., an energy value in a heart sound window defined by, among other things, a cardiac signal feature, one or more other heart sounds, or combinations thereof, over one or more cardiac cycles, etc.). In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform.

In an example, one or more of an initial atrial fibrillation detection, such as using cardiac electrical information, a confirmed atrial fibrillation detection using the determined indication of the atrial fibrillation S4 heart sound for the S4 signal portion based on the determined first and second correlations, or a composite detection using the cardiac electrical information and the determined indication of the atrial fibrillation S4 heart sound for the S4 signal portion based on the determined first and second correlations can transition operation of a medical device, such as from a low-power mode to a high-power mode. In certain examples, the high-power mode can be in contrast to the low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

In contrast, an initial atrial fibrillation detection can be rejected using a determined indication of the non-atrial fibrillation S4 heart sound (e.g., an S4 signal template in normal sinus rhythm, etc.).

Additionally, or alternatively, event storage can be triggered, such as in response to a detected or confirmed atrial fibrillation detection. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), such as in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, such as to increase arterial pressure, maintain cardiac output, and to disrupt or reduce the impact of the detected atrial fibrillation event.

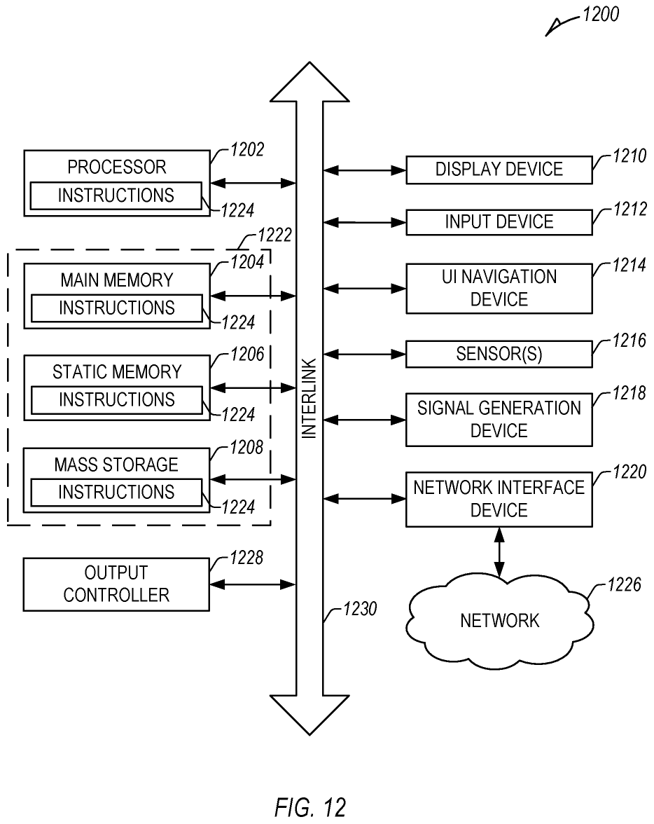

FIG. 12 illustrates a block diagram of an example machine 1200 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 1200. Circuitry (e.g., processing circuitry, an assessment circuit, etc.) is a collection of circuits implemented in tangible entities of the machine 1200 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1200 follow.

In alternative embodiments, the machine 1200 may operate as a standalone device or may be connected (e.g., networked) to other machines. In a networked deployment, the machine 1200 may operate in the capacity of a server machine, a client machine, or both in server-client network environments. In an example, the machine 1200 may act as a peer machine in peer-to-peer (P2P) (or other distributed) network environment. The machine 1200 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1200 may include a hardware processor 1202 (e.g., a central processing unit (CPU), a graphics processing unit (GPU), a hardware processor core, or any combination thereof), a main memory 1204, a static memory (e.g., memory or storage for firmware, microcode, a basic-input-output (BIOS), unified extensible firmware interface (UEFI), etc.) 1206, and mass storage 1208 (e.g., hard drive, tape drive, flash storage, or other block devices) some or all of which may communicate with each other via an interlink (e.g., bus) 1230. The machine 1200 may further include a display unit 1210, an input device 1212 (e.g., a keyboard), and a user interface (UI) navigation device 1214 (e.g., a mouse). In an example, the display unit 1210, input device 1212, and UI navigation device 1214 may be a touch screen display. The machine 1200 may additionally include a signal generation device 1218 (e.g., a speaker), a network interface device 1220, and one or more sensors 1216, such as a global positioning system (GPS) sensor, compass, accelerometer, or one or more other sensors. The machine 1200 may include an output controller 1228, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 1202, the main memory 1204, the static memory 1206, or the mass storage 1208 may be, or include, a machine-readable medium 1222 on which is stored one or more sets of data structures or instructions 1224 (e.g., software) embodying or utilized by any one or more of the techniques or functions described herein. The instructions 1224 may also reside, completely or at least partially, within any of registers of the hardware processor 1202, the main memory 1204, the static memory 1206, or the mass storage 1208 during execution thereof by the machine 1200. In an example, one or any combination of the hardware processor 1202, the main memory 1204, the static memory 1206, or the mass storage 1208 may constitute the machine-readable medium 1222. While the machine-readable medium 1222 is illustrated as a single medium, the term “machine-readable medium” may include a single medium or multiple media (e.g., a centralized or distributed database, and/or associated caches and servers) configured to store the one or more instructions 1224.

The term “machine-readable medium” may include any medium that is capable of storing, encoding, or carrying instructions for execution by the machine 1200 and that cause the machine 1200 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1224 may be further transmitted or received over a communications network 1226 using a transmission medium via the network interface device 1220 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 1220 may include one or more physical jacks (e.g., Ethernet, coaxial, or phone jacks) or one or more antennas to connect to the communications network 1226. In an example, the network interface device 1220 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1200, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system, comprising:
a signal receiver circuit configured to receive (1) cardiac electrical information of a patient, including a timing metric between first and second cardiac features of the patient; and (2) cardiac acceleration information of the patient, including a fourth heart sound (S4) signal portion occurring between the first and second cardiac features of the patient;
an assessment circuit configured to determine a first correlation of a morphology of the S4 signal portion to a non-atrial fibrillation S4 template and a second correlation of a morphology of the S4 signal portion to an atrial fibrillation S4 template, and to determine an indication of one of an atrial fibrillation S4 heart sound or a non-atrial fibrillation S4 heart sound for the S4 signal portion based on the determined first and second correlations; and
an atrial fibrillation detection circuit configured to detect an atrial fibrillation event of the patient using the received timing metric and the determined indication for the S4 heart sound portion.

2. The medical device system of claim 1, comprising:
a cardiac sensor, coupled to the signal receiver circuit, configured to sense the cardiac electrical information of the patient; and
a heart sound sensor, coupled to the signal receiver circuit, configured to sense the cardiac acceleration information of the patient.

3. The medical device system of claim 2, comprising an implantable medical device comprising the electrical sensor, the heart sound sensor, the signal receiver circuit, the assessment circuit, and the atrial fibrillation detection circuit.

4. The medical device system of claim 2, wherein the cardiac sensor comprises a processing circuit configured to determine the timing metric between the first and second cardiac features of a first cardiac interval of the patient or between first and second cardiac features of respective successive first and second cardiac intervals of the patient, and
wherein the heart sound sensor comprises a processing circuit configured to determine the S4 signal portion in an S4 window of the cardiac acceleration information of the corresponding first or second cardiac interval of the patient.

5. The medical device system of claim 1, wherein the first and second cardiac features include equivalent detected features in successive first and second cardiac intervals.

6. The medical device system of claim 5, wherein the first cardiac feature is an R wave of a first cardiac interval, and
wherein the second cardiac feature is an R wave of an ensuing second cardiac interval.

7. The medical device system of claim 1, wherein the timing metric is a timing metric of a cardiac interval, and wherein the S4 signal portion is an S4 signal portion of the cardiac interval.

8. The medical device system of claim 1, wherein the atrial fibrillation event comprises multiple cardiac intervals, and
wherein the assessment circuit is configured to determine the indication of one of the atrial fibrillation S4 heart sound or the non-atrial fibrillation S4 heart sound for the S4 signal portion based on the determined first and second correlations for each of the multiple cardiac intervals.

9. The medical device system of claim 1, wherein the atrial fibrillation event comprises multiple cardiac intervals,
wherein the assessment circuit is configured to determine a composite S4 signal portion using S4 signal portions occurring over the multiple cardiac intervals,
wherein the assessment circuit is configured to determine the first correlation between the composite S4 signal portion and the non-atrial fibrillation S4 template and the second correlation between the composite S4 signal portion and the atrial fibrillation S4 heart sound, and
wherein the assessment circuit is configured to determine the indication of one of the atrial fibrillation S4 heart sound or the non-atrial fibrillation S4 heart sound for the composite S4 signal portion based on the determined first and second correlations.

10. The medical device system of claim 1, wherein the assessment circuit is configured to determine the indication of one of the atrial fibrillation S4 heart sound or the non-atrial fibrillation S4 heart sound for the S4 heart sound in the S4 signal portion based on a difference between a correlation of the S4 signal portion to the non-atrial fibrillation S4 template and a correlation of the S4 signal portion to the atrial fibrillation S4 template.

11. The medical device system of claim 10, wherein the assessment circuit is configured to determine the indication of one of the atrial fibrillation S4 heart sound or the non-atrial fibrillation S4 heart sound for the S4 heart sound in the S4 signal portion based on a difference of the correlation of the S4 signal portion the atrial fibrillation S4 template from the correlation of the S4 signal portion to the non-atrial fibrillation S4 template.

12. A method comprising:
receiving, at a signal receiver circuit, cardiac electrical information of a patient, including a timing metric between first and second cardiac features of the patient;
receiving, at the signal receiver circuit, cardiac acceleration information of the patient, including a fourth heart sound (S4) signal portion occurring between the first and second cardiac features of the patient;
determining, using an assessment circuit, a first correlation of a morphology of the S4 signal portion to a non-atrial fibrillation S4 template and a second correlation of the morphology of the S4 signal portion to an atrial fibrillation S4 template;
determining, using the assessment circuit, an indication of one of an atrial fibrillation S4 heart sound or a non-atrial fibrillation S4 heart sound for the S4 signal portion based on the determined first and second correlations; and
detecting, using an atrial fibrillation detection circuit, an atrial fibrillation event of the patient using the received timing metric and the determined indication for the S4 signal portion.

13. The method of claim 12, comprising:
sensing the cardiac electrical information of the patient using a cardiac sensor coupled to the signal receiver circuit; and
sensing the cardiac acceleration information of the patient using a heart sound sensor coupled to the signal receiver circuit.

14. The method of claim 13, wherein the electrical sensor, the heart sound sensor, the signal receiver circuit, the assessment circuit, and the atrial fibrillation detection circuit are components of an implantable medical device.

15. The method of claim 13, comprising:
determining, using a processing circuit of the cardiac sensor, the timing metric between the first and second cardiac features of a first cardiac interval of the patient or between first and second cardiac features of respective successive first and second cardiac intervals of the patient; and
determining, using a processing circuit of the heart sound sensor, the S4 signal portion in an S4 window of the cardiac acceleration information of the corresponding first or second cardiac interval of the patient.

16. The method of claim 12, wherein the first and second cardiac features include equivalent detected features in successive first and second cardiac intervals.

17. The method of claim 12, wherein the timing metric is a timing metric of a first cardiac interval, and wherein the S4 signal portion is an S4 signal portion of the first cardiac interval.

18. The method of claim 12, wherein the atrial fibrillation event comprises multiple cardiac intervals, and
wherein determining the indication of one of the atrial fibrillation S4 heart sound or the non-atrial fibrillation S4 heart sound for the S4 signal portion includes based on the determined first and second correlations for each of the multiple cardiac intervals.

19. The method of claim 12, wherein the atrial fibrillation event comprises multiple cardiac intervals, wherein the method includes:
determining a composite S4 signal portion using S4 signal portions occurring over the multiple cardiac intervals,
wherein determining the first correlation comprises between the composite S4 signal portion and the non-atrial fibrillation S4 template,
wherein determining the second correlation comprises between the composite S4 signal portion and the atrial fibrillation S4 heart sound, and
wherein determining the indication of one of the atrial fibrillation S4 heart sound or the non-atrial fibrillation S4 heart sound comprises for the composite S4 signal portion based on the determined first and second correlations.

20. The method of claim 12, wherein determining the indication of one of the atrial fibrillation S4 heart sound or the non-atrial fibrillation S4 heart sound for the S4 heart sound in the S4 signal portion comprises based on a difference between a correlation of the S4 signal portion to the non-atrial fibrillation S4 template and a correlation of the S4 signal portion to the atrial fibrillation S4 template.
